(19) United States
(12) Patent Application Publication
(10) Pub. No.: US 2017/0007257 A1
(43) Pub. Date: Jan. 12, 2017

(54) UMBILICAL CORD CLAMP

(71) Applicant: UWM Research Foundation, Inc., Milwaukee, WI (US)

(72) Inventors: Charles Potter, Mequon, WI (US); Ilya Avdeev, Shorewood, WI (US); Maxwell Potter, Mequon, WI (US); Alex Francis, Kenosha, WI (US)

(21) Appl. No.: 15/204,701

(22) Filed: Jul. 7, 2016

Related U.S. Application Data
(60) Provisional application No. 62/190,387, filed on Jul. 9, 2015.

(51) Int. Cl. A61B 17/122

(52) U.S. Cl. CPC A61B 17/122; A61B 2017/00438

(57) ABSTRACT

A clamping device for an umbilical cord includes a first member having a first clamping surface and a second member having a second clamping surface. The second member is flexibly coupled to the first member. The clamping device further includes an arcuate body extending away from the first member to assist an operator in moving the first member relative to the second member. The arcuate body is removably coupled to the first member.

### RELATED APPLICATIONS

The present invention claims priority to U.S. Provisional Application No. 62/190,387, filed Jul. 9, 2015, the entire contents of which are hereby incorporated by reference.

### FIELD OF THE INVENTION

The present invention relates to medical clamps, and more particularly, to umbilical cord clamps.

### BACKGROUND

Hospitals facilitate child birth in a clean environment with sterile equipment. Sterile and non-sterile clamps have many applications in hospital settings. For example, sterile clamps are used on umbilical cords to restrict blood flow when the umbilical cord is cut during child birth. These types of clamps reduce the amount of blood loss from the mother and the infant.

While blood flow from the umbilical cord naturally decreases over the course of a few minutes, complications during child birth may require immediate medical care of the infant. Typically, to provide more immediate medical care of the infant, the umbilical cord is clamped and cut.

### SUMMARY

In one embodiment, the invention provides a clamping device for an umbilical cord. The clamping device includes a first member having a first clamping surface and a second member having a second clamping surface. The second member is flexibly coupled to the first member. The clamping device further includes an arcuate body extending away from the first member to assist an operator in moving the first member relative to the second member. The arcuate body is removably coupled to the first member.

In another embodiment, the invention provides a clamping device for an umbilical cord. The clamping device includes a first member having a first clamping surface and a second member having a second clamping surface. The second member is flexibly coupled to the first member about a pivot axis. The clamping device further includes a finger retainer to assist an operator in pivoting the first member relative to the second member.

In yet another embodiment, the invention provides a method of clamping an umbilical cord with a clamping device. The method includes receiving a force through a finger retainer of the clamping device in a first direction to engage a first member with a second member. The first member has a first clamping surface and the second member has a second clamping surface. The second member is flexibly coupled to the first member. The method further includes coupling the first member to the second member such that the first member remains engaged with the second member. The method further includes receiving a force through the finger retainer in a second direction different from the first direction to remove the finger retainer from the clamping device.

Other aspects of the invention will become apparent by consideration of the detailed description and accompanying drawings.

### BRIEF DESCRIPTION OF THE DRAWINGS

FIG. 1 is a perspective view of an umbilical cord clamp in accordance with an embodiment of the invention, illustrating the umbilical cord clamp in an open position.

FIG. 2 is a perspective view of the umbilical cord clamp of FIG. 1 in a closed position.

FIG. 3 is a side view of the umbilical cord clamp of FIG. 1, illustrating a first member coupled to a second member and a finger retainer coupled to each of the first and second members.

FIG. 4A is a cross-sectional view of the umbilical cord clamp along section line 4-4.

FIG. 4B is an enlarged cross-sectional view of the umbilical cord clamp of FIG. 4A.

FIG. 5A is a cross-sectional view of the umbilical cord clamp along section line 5-5.

FIG. 5B is an enlarged cross-sectional view of the umbilical cord clamp of FIG. 5A.

FIG. 6 is a partial cross-sectional view of the umbilical cord clamp, illustrating the umbilical cord clamp in the closed position with the finger retainers removed from the first and second members.

FIG. 7 is a perspective view of an umbilical cord clamp in accordance with another embodiment of the invention, illustrating the umbilical cord clamp in an open position.

FIG. 8 is a perspective view of the umbilical cord clamp of in FIG. 7 a closed position.

FIG. 9 is a side view of the umbilical cord clamp of FIG. 7, illustrating a first member coupled to a second member and a finger retainer coupled to each of the first and second members.

FIG. 10A is a cross-sectional view of the umbilical cord clamp along section line 10-10.

FIG. 10B is an enlarged cross-sectional view of the umbilical cord clamp of FIG. 10A.

FIG. 11A is a cross-sectional view of the umbilical cord clamp along section line 11-11.

FIG. 11B is an enlarged cross-section view of the umbilical cord clamp of FIG. 11A.

FIG. 12 is a partial cross-sectional view of the umbilical cord clamp, illustrating the umbilical cord clamp in the closed position with the finger retainers removed from the first and second members.

FIG. 13 is a perspective view of an umbilical cord clamp in accordance with another embodiment of the invention.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

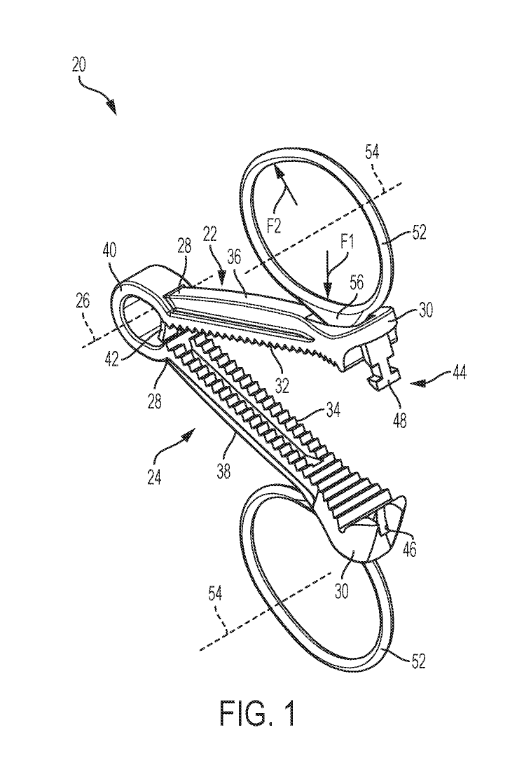

FIG. 1 illustrates an umbilical cord clamp 20. The umbilical cord clamp 20 is a medical clamping device including a first member 22 and a second member 24 that cooperatively work together to clamp an umbilical cord to inhibit blood flow within the umbilical cord. The first member 22 pivots relative to the second member 24, or vice versa, about a pivot axis 26. The pivot axis 26 is disposed proximate a first end 28 of the clamp 20 and each member 22, 24 extends away from the first end 28 toward a second end 30 of the clamp 20. The first end 28 may also be referred to as a pivot end, and the second end 30 may also be referred to as a distal end. The clamp 20 of the illustrated embodiment is formed as a monolithic component via a single casting or other suitable process. In other embodiments, the clamp 20 may be formed as multiple pieces via machining, casting or other manufacturing processes.

With continued reference to FIG. 1, the first member 22 includes a first clamping surface 32 and the second member 24 includes a second clamping surface 34. Generally, the first and second clamping surfaces 32, 34 face each other, whereas the first and second members 22, 24 also include outer surfaces 36, 38 that generally face away from each other. As illustrated, the first and second clamping surfaces 32, 34 have serrated, interlocking teeth to provide additional grip and friction against the umbilical cord. As such, the clamp 20 is inhibited from movement relative to the umbilical cord. In other embodiments, the first and second clamping surfaces 32, 34 may alternatively be flat, formed with other geometric structures (i.e., bumps, hollows, etc.), provided with a material having a high coefficient of friction, or a combination thereof.

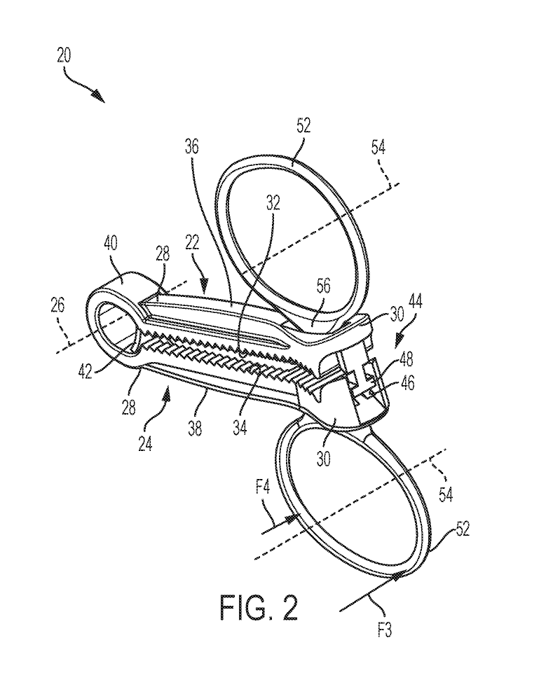

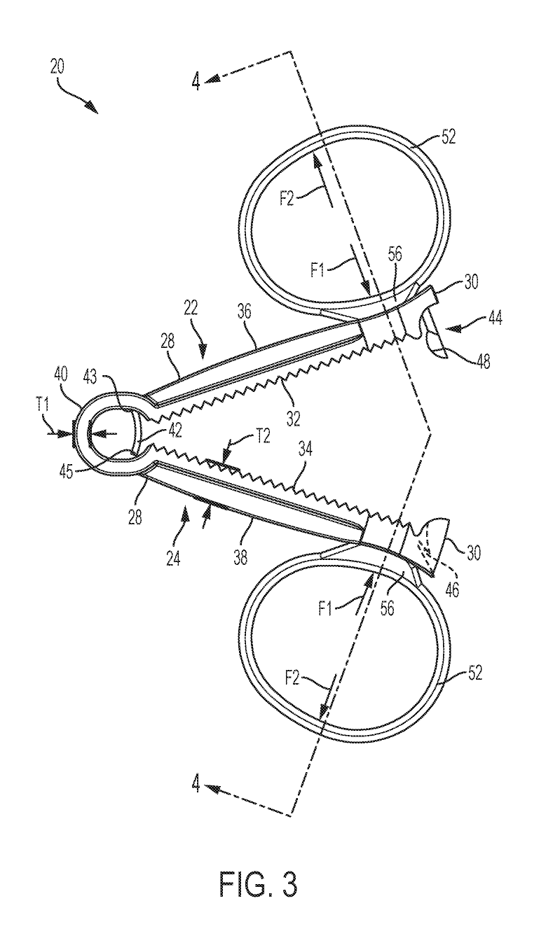

With reference to FIGS. 1 and 2, the clamp 20 further includes a joint 40 disposed at the first end 28 of the clamp 20 that flexibly couples the first member 22 to the second member 24. The joint 40 is configured as a thin-walled member that is circumferentially disposed about the pivot axis 26. As shown in FIG. 3, the thickness T1 of the material making up the joint 40 (i.e., measured in a direction perpendicular to the pivot axis 26) is less than the thickness T2 of the material making up the first and second members 22, 24 measured between the clamping surface 32 (or 34) and the outer surface 36 (or 38). The joint 40 enables the first member 22 and the second member 24 to pivot relative to each other between an open position (FIG. 1) and a closed position (FIG. 2). In the open position, the first member 22 is angularly spaced apart from the second member 24 such that the clamp 20 is generally V-shaped. In contrast, the first member 22 is generally parallel to the second member 24 in the closed position. In some cases, at least a portion of the first clamping surface 32 engages a portion of the second clamping surface 34 in the closed position. The clamp 20 further includes a biasing member 42 disposed at the first end 28 of the clamp 20. As shown in FIG. 3, the biasing member 42 includes a connecting end 43 that is coupled to a portion of the joint 40 and a free end 45 that extends away and is cantilevered from the connecting end 43. The free end 45 engages an inner periphery of the joint 40. The biasing member 42 urges the clamp 20 toward the open position and the biasing member 42 deforms when the clamp 20 is in the closed position. In addition, the material making up the joint 40 is resilient, thereby providing assistance in urging the clamp 20 toward the open position.

With reference to FIGS. 1-3, the clamp 20 further includes a locking mechanism 44 disposed at the second end 30 of the clamp 20 to selectively couple the first member 22 to the second member 24. The locking mechanism 44 includes a slot 46 formed as a part of the second member 24 and a projection 48 formed as a part of the first member 22. The projection 48 generally extends toward the slot 46. The locking mechanism 44 is operable between a locked state (FIG. 2), in which the clamp 20 is maintained in the closed position, and an unlocked state (FIG. 1), in which the clamp 20 is in the open position. In the closed position, the projection 48 is received within and couples to the slot 46. Various engagement mechanisms may be implemented. For example, with respect to FIGS. 1 and 2, the projection includes two hooks extending away from the projection 48 that are received within and couple to corresponding recesses of the slot 46. Although generally rigid, the projection 48 may flex under force to permit locking and unlocking the hooks and recesses. In other embodiments, the locking mechanism 44 can be other types of quick-connect-disconnect mechanisms.

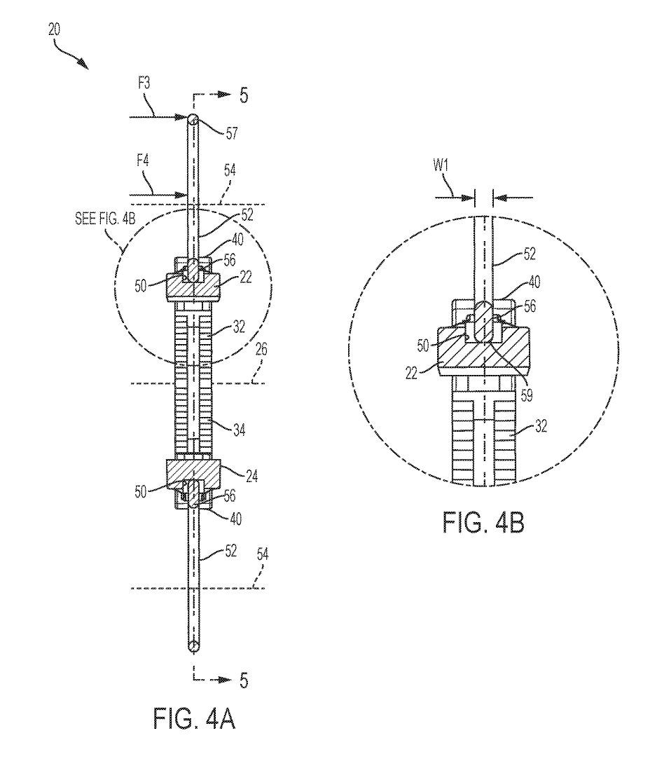

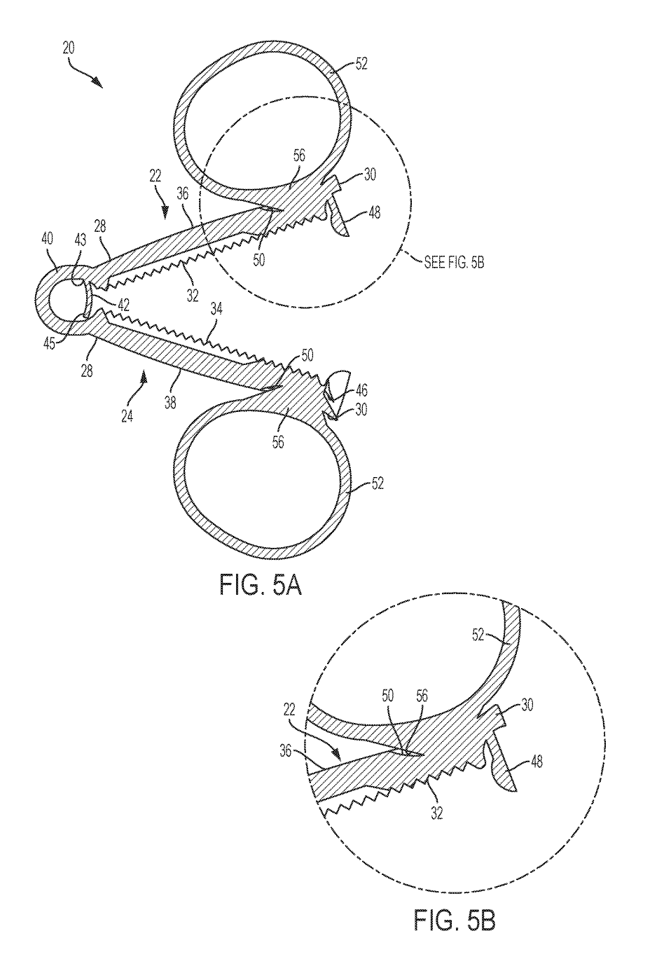

With reference to FIGS. 4A-5B, the first and second members 22, 24 each include a recess 50 formed in the outer surface 36, 38. In the illustrated embodiment, each recess 50 is formed as a concavity within each member 22, 24. Specifically, the recess 50 is formed such that no portion of the recess 50 extends beyond the outer surfaces 36, 38 of the first and second members 22, 24.

With reference to FIGS. 1-5B, the first and second members 22, 24 each include a finger retainer 52 to receive at least one finger of an operator. The finger retainers 52 extend away from the corresponding members 22, 24 to assist the operator in moving the first member 22 relative to the second member 24. The finger retainers 52 are formed as arcuate bodies. In the illustrated embodiment, the arcuate body of each finger retainer 52 forms a loop, which is in the shape of an oblong circle. Each finger retainer 52 defines a finger axis 54 (also referred to as an arcuate body axis) that is parallel to the pivot axis 26. As such, each finger retainer 52 is oriented generally parallel to the corresponding member 22, 24. Each finger retainer 52 includes a base 56 that is coupled to each member 22, 24 within the recess 50 of each respective member 22, 24, as shown in FIG. 4B. Although the finger retainers 52 of the illustrated embodiment are oriented parallel to the first and second members 22, 24, in other embodiments, the finger retainers 52 may alternatively be oriented perpendicular (i.e., such that the axis 26 is perpendicular to the axes 54) or at another angular offset relative to the first and second members 22, 24.

In the illustrated embodiment, each finger retainer 52 generally has circular cross section 57 (FIG. 4A) but can alternatively have a rectangular or oblong cross section to vary surface area for the operator to contact the clamp 20, vary pressure on operator fingers, and account for operator preferences. Although integrally formed with the clamp 20, the finger retainers 52 are removable from the clamp 20. Particularly, the connection between the recess 50 of the members 22, 24 and the base 56 of the finger retainers 52 is breakable due in part to the a curved surface 59 of the base 56 being tangentially connected to the recess 50 (FIG. 4B). Accordingly, the finger retainers 52 are removably coupled to the members 22, 24. In other embodiments, the finger retainers 52 may alternatively be removably coupled to the members 22, 24 via a screw joint, a slip joint, or other types of quick-release mechanical joints.

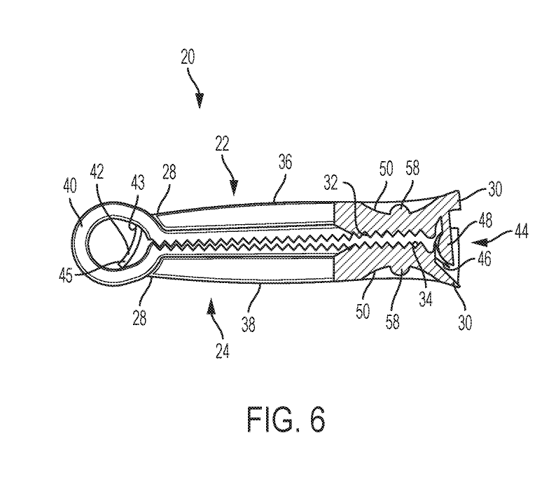

With reference to FIG. 6, when the finger retainer 52 is removed from the corresponding member 22, 24, a burr 58 is formed as residual material where the connection between the recess 50 and the finger retainer 52 once was. As shown, the burr 58 does not extend beyond the outer surfaces 36, 38 of the first and second members 22, 24. The burr 58 remains disposed within the recess 50 to inhibit skin irritation if the outer surface 36, 38 comes in contact with skin.

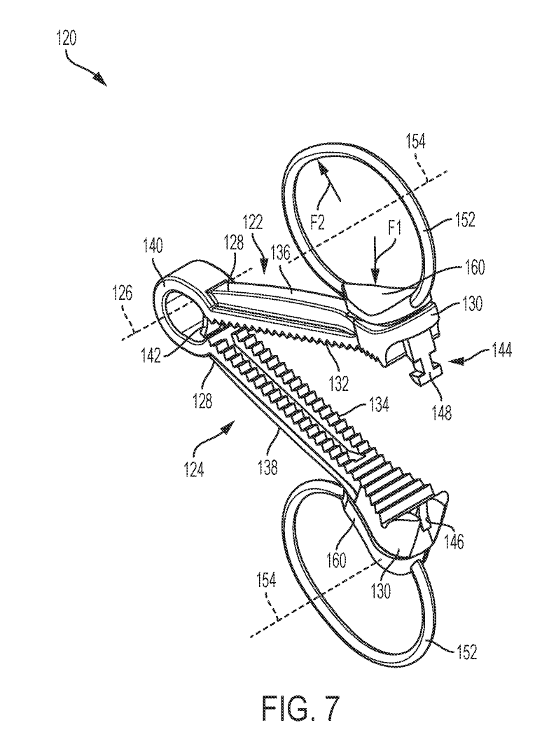

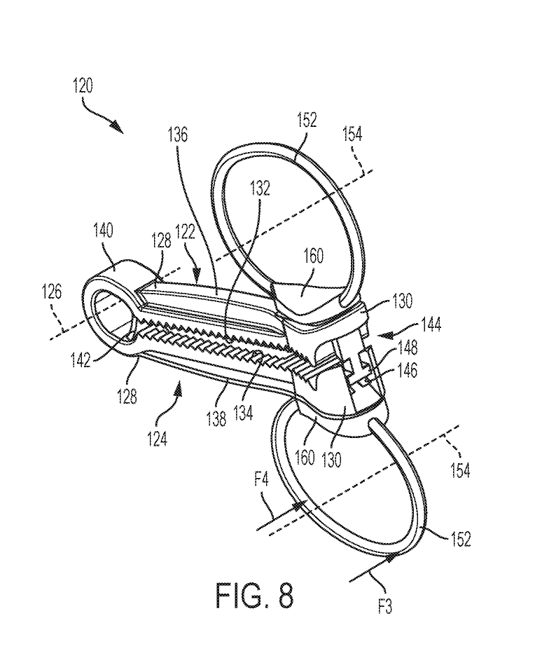

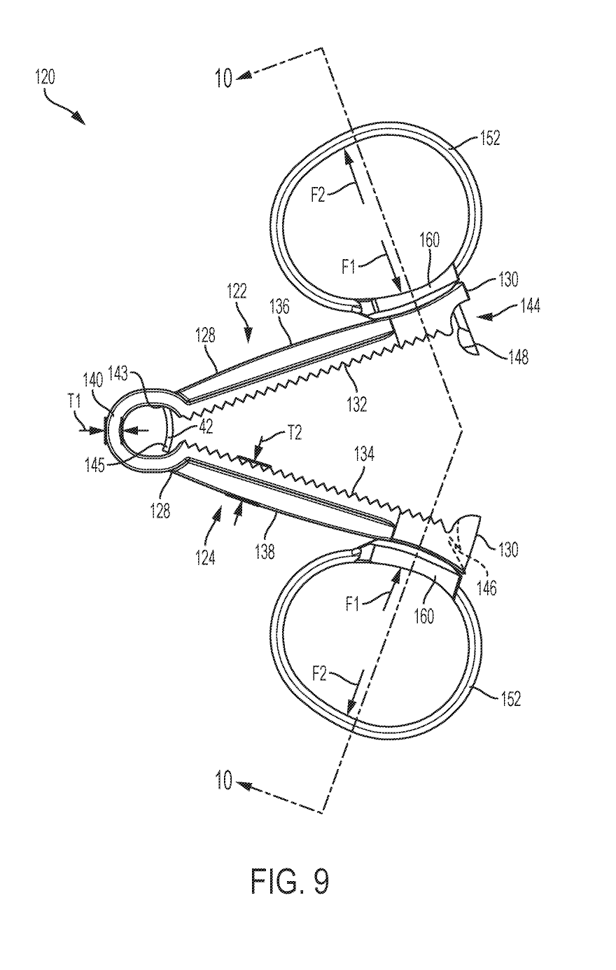

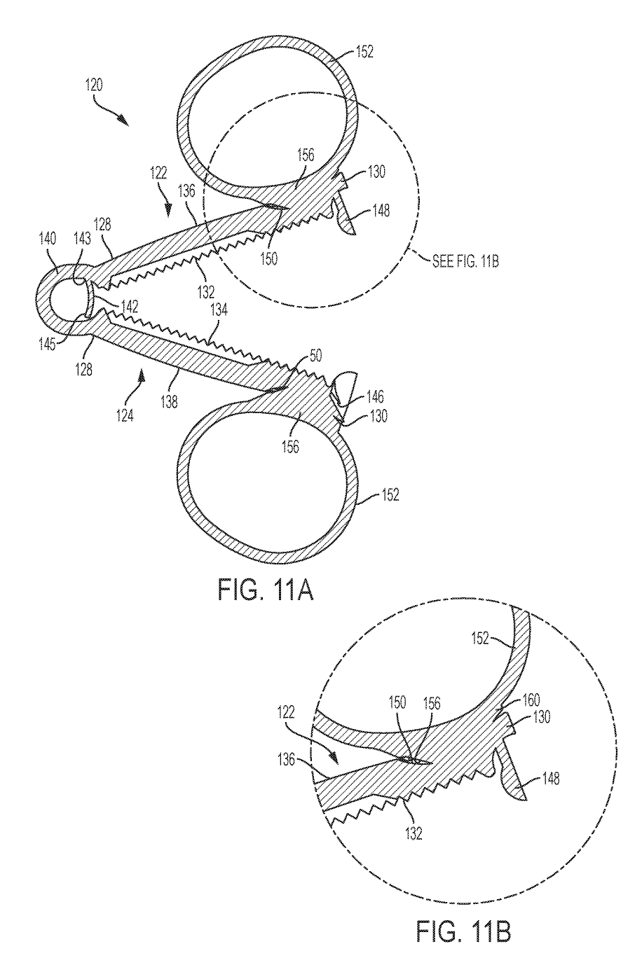

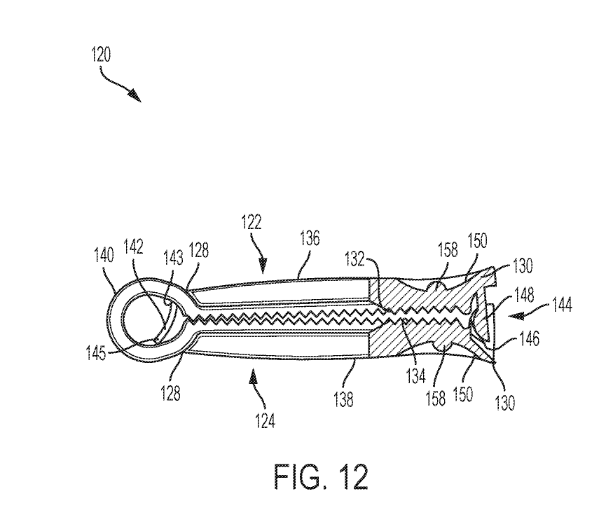

FIGS. 7-12 illustrate an umbilical cord clamp 120 in accordance with another embodiment. The clamp 120 includes a gripping region 160, but is otherwise generally similar to the clamp 20 described above with reference to FIGS. 1-6, with like components being shown with like reference numerals plus 100. Only some differences between the clamps 20, 120 are described below.

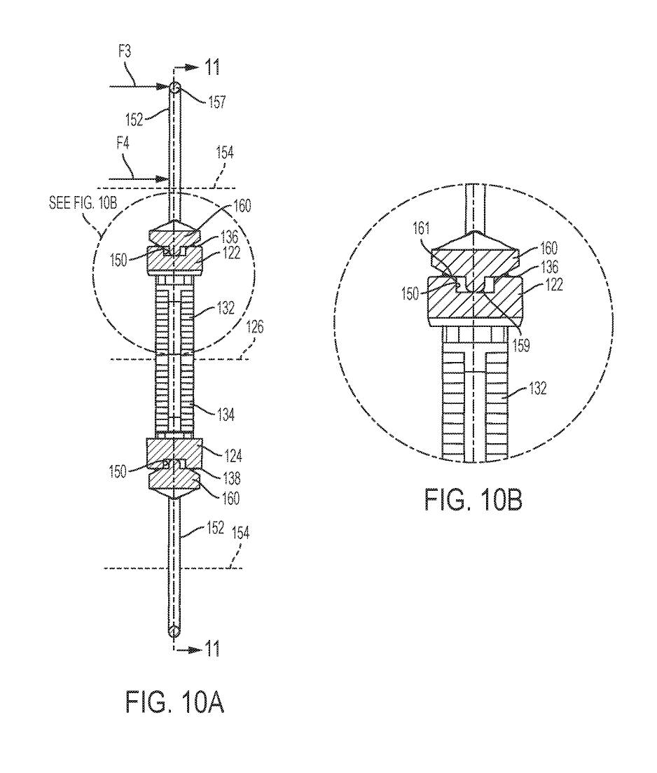

Each finger retainer 152 of the clamp 120 includes the gripping region 160, as shown in FIGS. 7-10B. The gripping region 160 is disposed adjacent the outer surface 136, 138 of each member 122, 124 and has a larger thickness measured along a direction parallel to the finger axis 154 than a thickness of a remainder of the finger retainers 152. The gripping region 160 provides a broad surface area for the operator to engage. As such, the gripping region 160 minimizes pressure to the fingers of the operator and facilitates manipulation of the clamp 120. Furthermore, at least a portion of the gripping region 160 is in contact with the outer surface 136, 138 of each member 122, 124 (FIG. 10B) to provide additional stabilization to the finger retainers 152 when the clamp 120 is being moved from the open position to the closed position. For example, overhang portion 161 abuts the outer surface 136. In other words, the gripping regions 160 and the overhand portion 161 stabilize the finger retainers 152 to decrease the likelihood of inadvertent removal or flexing of the finger retainers 152.

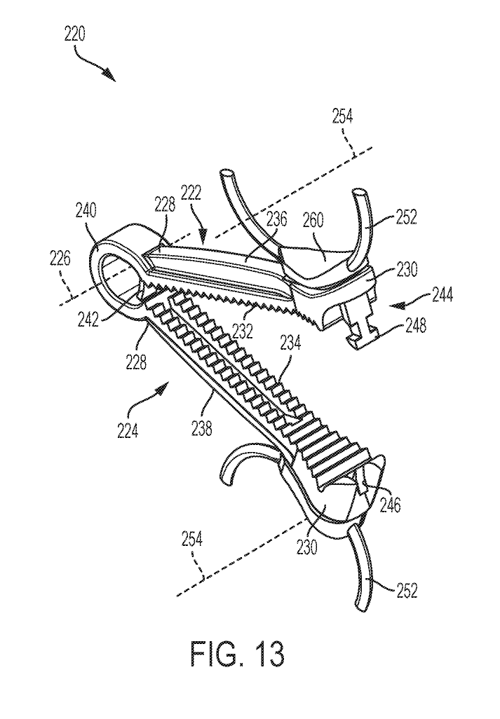

FIG. 13 illustrates an umbilical cord clamp 220 in accordance with another embodiment. The clamp 220 includes a finger retainer 252 and a gripping region 260, but is otherwise generally similar to the clamp 20 described above with reference to FIGS. 1-6, with like components being shown with like reference numerals plus 200. Only some differences between the clamps 20, 220 are described below.

As illustrated, the finger retainers 252 are formed as arcuate bodies. However, in this case, the arcuate bodies are not formed as complete loops. Rather, the finger retainers 252 are C-shaped such that each finger retainer 252 defines only a portion of a loop. In other embodiments, the finger retainers 252 may be formed geometrically different. The gripping region 260 is generally similar to the gripping region 160 as described above with respect to FIGS. 7-12.

A method of operating the umbilical cord clamp 20 of FIGS. 1-6 is discussed below. While the method is described with respect to clamp 20, the method is similarly applicable to the clamps 120 and 220 a well. In operation, the fingers of the operator are inserted into the finger retainers 52 along the finger axis 54. At this point, the operator positions the clamp 20 around the umbilical cord such that the first and second clamping surfaces 32, 34 are adjacent the umbilical cord. Subsequently, the clamp 20 is moved from the open position, in which the locking mechanism 44 is in the unlocked state, toward the closed position, in which the locking mechanism 44 is in the locked state. Specifically, the operator applies a force F1 (FIGS. 1 and 3) in a direction perpendicular to the outer surface 36, 38 of the first and second members 22, 24 to pivot the first member 22 relative to the second member 24. The force F1 applied to the first and second members 22, 24 is sufficient to overcome the biasing member 42 and pinching the umbilical cord between the first and second clamping surfaces 32, 34 to reach the locked state.

Once the clamp 20 is secured to the umbilical cord in the locked state, the finger retainers 52 are no longer required and are removed from the first and second members 22, 24. Essentially, the base 56 of the finger retainers 52 enables compression forces (e.g., the force F1) to be exerted through the finger retainers 52 without separating the finger retainers 52 from the first and second members 22, 24. However, a tension force F2 (FIG. 3), a bending force F3 (FIG. 4A), or twisting force F4 (FIG. 4A) exerted on the finger retainers 52 results in easy removal of the finger retainers 52 from the first and second members 22, 24. The forces F3, F4 (also shown in FIG. 2) are exerted on the finger retainers 52 in a direction parallel to the finger axis 54, thereby resulting in a moment force at the interface between the base 56 and the recess 50. As such, the finger retainers 52 are especially removed with ease from the first and second members 22, 24 when the forces F3, F4 are applied to the finger retainers 52. In other words, the finger retainers 52 are easily removed from the members 22, 24 when a torque is applied to the finger retainers 52. As a result of the tangential connection between the curved surface 59 of the base 56 and the recess 50 of the members 22, 24, a weak zone is formed to control the break point. As illustrated, the curved surface 59 of the base 56 of the finger retainer 52 has a width W1 (FIG. 4B) that is greater than the width of the interface between the finger retainer 52 and the recess 50. Accordingly, the finger retainers 52 and interface at the recess 50 are designed such, when the forces (e.g., F2, F3, or F4) are applied to the finger retainer 52, the forces cause the break point to be at the weak zone (i.e., at the interface), rather than another part of the finger retainers 52. Also, the tangential connection minimizes the amount of material at the interface between the finger retainers 52 and the members 22, 24, thereby minimizing the size of the burr 58. As such, when the finger retainers 52 are removed from the clamp 20, the burr 58 is completely disposed within the recess 50 such that no portion of the burr 58 extends beyond the upper surface of the members 22, 24. In other embodiments, the width W1 of the curved surface 59 is equal to or greater than the width of the interface between the finger retainer 52 and the recess 50, but the interface is nevertheless a weak zone at which the finger retainers 52 will break away under force (e.g., forces F2, F3, or F4).

Although embodiments discussed above include finger retainers 52, 152, and 252 that are removable, in some embodiments, the finger retainers 52, 152, and 252 are not removably coupled but, rather, are intended to remain on the clamp even after clamping on the umbilical cord. For example, in such embodiments, the interface between the finger retainers 52, 152, and 252 may include more material coupling the finger retainers to the first and second members than illustrated, for instance, in FIGS. 4B and 10B. As an example, with reference to FIGS. 4B, additional material may fill in all or part of the recess 50 to join the sides of the finger retainer 52 to the first member 22. Accordingly, such interfaces for finger retainers that are not removably coupled do not include a weak zone minimizing the amount of material at the interface between the finger retainers and the members.

Thus, embodiments of the invention provide, among other things, a removable finger retainer for an umbilical cord clamp. Sterile equipment is used during child birth due to the fluids present during child birth. However, several factors (e.g., wearing medical gloves, fast paced medical operations, etc.) can lead to mishandling of clamps that do not include finger retainers, thereby resulting in an unsterile clamp that can no longer be applied to the umbilical cord. Embodiments of the invention aid the operator in one or more of removing the clamp from its packaging, handling the clamp, placing the clamp on the umbilical cord, applying a force at the free end of the clamp to secure the umbilical cord, and placing the clamp in a locked position. Various features and advantages of the invention are set forth in the following claims.

1. A clamping device for an umbilical cord, the clamping device comprising:
a first member having a first clamping surface;
a second member having a second clamping surface, the second member is flexibly coupled to the first member; and
an arcuate body extending away from the first member to assist an operator in moving the first member relative to the second member, the arcuate body is removably coupled to the first member.

2. The clamping device of claim 1, further comprising a recessed region formed in the first member, wherein the arcuate body is at least partially disposed within the recessed region.

3. The clamping device of claim 2, wherein a portion of the arcuate body remains disposed within the recessed region when the arcuate body is removed from the first member such that the portion of the arcuate body does not extend beyond the recessed region.

4. The clamping device of claim 1, further comprising a locking mechanism operative between an unlocked state, in which the first and second clamping surfaces are spaced away from each other and a locked state, in which the first and second clamping surfaces remain in contact.

5. The clamping device of claim 1, further comprising a first end defining a pivot axis and a second end opposite the first end, wherein the arcuate body is disposed proximate the second end.

6. The clamping device of claim 5, wherein the arcuate body defines an arcuate body axis that is parallel to the pivot axis.

7. The clamping device of claim 6, wherein the arcuate body is removed from the first member when a force is applied to the arcuate body in a direction parallel to the arcuate body axis.

8. The clamping device of claim 5, wherein the first member and the second member pivot about the pivot axis when the first member moves relative to the second member.

9. The clamping device of claim 1, wherein the arcuate body is a first arcuate body and the clamping device further includes a second arcuate body removably coupled to the second member.

10. The clamping device of claim 1, wherein the first clamping surface and the second clamping surface are both serrated and the arcuate body defines a loop.

11. A clamping device for an umbilical cord, the clamping device comprising:
a first member having a first clamping surface;
a second member having a second clamping surface, the second member is flexibly coupled to the first member about a pivot axis; and
a finger retainer to assist an operator in pivoting the first member relative to the second member.

12. The clamping device of claim 11, further comprising a recessed region formed in the first member, wherein the finger retainer is at least partially disposed within the recessed region and is removably coupled to the first member.

13. The clamping device of claim 12, wherein a portion of the finger retainer remains disposed within the recessed region when the finger retainer is removed from the first member such that the portion of the finger retainer does not extend beyond the recessed region.

14. The clamping device of claim 11, further comprising a locking mechanism operative between an unlocked state, in which the first and second clamping surfaces are spaced away from each other and a locked state, in which the first and second clamping surfaces remain in contact.

15. The clamping device of claim 11, wherein the finger retainer defines an arcuate body.

16. The clamping device of claim 15, wherein the finger retainer has a base with a curved surface that is tangentially connected to the first member such that applying a torque to the finger retainer removes the finger retainer from the first member.

17. The clamping device of claim 11, wherein the finger retainer is a first finger retainer and the clamping device further includes a second finger retainer removably coupled to the second member.

18. The clamping device of claim 17, wherein the first and second finger retainers each include a gripping region having a larger thickness measured along a direction parallel to the pivot axis than a thickness of a remainder of the first and second finger retainers.

19. A method of clamping an umbilical cord with a clamping device, the method comprising:
receiving a force through a finger retainer of the clamping device in a first direction to engage a first member with a second member, the first member has a first clamping surface and the second member has a second clamping surface, the second member is flexibly coupled to the first member;
coupling the first member to the second member such that the first member remains engaged with the second member; and
receiving a force through the finger retainer in a second direction different from the first direction to remove the finger retainer from the clamping device.

20. The method of claim 19, wherein receiving the force through the finger retainer in the first direction includes clamping the umbilical cord.
